(19) United States
(12) Patent Application Publication
(10) Pub. No.: US 2016/0066798 A1
(43) Pub. Date: Mar. 10, 2016

(54) Methods and Systems for Determining Hemodynamic Properties of a Tissue

(71) Applicant: UNIVERSITY OF WASHINGTON THROUGH ITS CENTER FOR COMMERCIALIZATION, Seattle, WA (US)

(72) Inventors: Ruikang K. WANG, Seattle, WA (US); Siavash YOUSEFI, Seattle, WA (US)

(21) Appl. No.: 14/781,677

(22) PCT Filed: Apr. 8, 2014
(86) PCT No.: PCT/US2014/033297

§ 371 Date: Oct. 1, 2015

Related U.S. Application Data
(60) Provisional application No. 61/810,096, filed on Apr. 9, 2013.

(51) Int. Cl. A61B 5/026; A61B 5/00

(52) U.S. Cl. CPC A61B 5/0261; A61B 5/4866; A61B 5/4836; A61B 5/7275; A61B 2019/5234

(57) ABSTRACT

Systems and methods for determining hemodynamic properties in a sample of a subject are provided. A system obtains one or more spectral interference signals from the sample during one or more scans, separates the spectral interference signals concerning tissue motion, cell motion, and noise within the sample by decomposing the tissue motion, the cell motion, and the noise into orthogonal basis functions. The system then determines hemodynamic properties of the sample from the separated cell motion. The system and method may be used for diagnosing, providing a prognosis, or monitoring treatment of a disorder of the sample.

### CROSS-REFERENCE TO RELATED APPLICATIONS

This application claims priority to U.S. Provisional Patent Application Ser. No. 61/810,096 filed on Apr. 9, 2013, which is hereby incorporated by reference in its entirety.

### BACKGROUND

Quantification and visualization of blood flow in various living tissues provides important information for diagnostics, treatment, and/or management of pathological conditions.

Hemodynamic visualization and quantification in micro-vessels and capillaries within tissues may be assessed to diagnose, treat, and monitor a number of pathological conditions, such as glaucoma, cancer, stroke, and a number of other disorders involving vascular components, for example, disorders of the brain, renal region, and skin. Such assessments may be used to provide guidance in medical, laser, or surgical management for a disorder of the tissue.

Hemodynamic visualization and quantification may also serve to measure and image blood flux within capillaries and small vessels. Blood flux as used herein is the number of blood cells that pass through a single capillary vessel per unit time. The microcirculatory system, including cardiovascular and lymphatic systems, has the important role of transporting oxygen, nutrition, fluid, and signaling molecules to living cells via arteries and collecting carbon dioxide and waste materials from the tissue cells. Thus, measuring and imaging blood flux within capillaries and small vessels may be assessed to diagnose, treat, and monitor a number of pathological conditions, such as vasculitis, angiogenesis, diabetes, cancer, cardiovascular, neurovascular, and retinal disease.

Techniques have been developed that attempt to visualize and quantify hemodynamic properties in micro-vessels and capillaries. Such techniques are not capable of dynamically estimating and separating moving tissues from stationary tissues, however, and further often require a static high-pass filter. Moreover, the total scanning time for three-dimensional in vivo applications is relatively long and the flow estimation is highly sensitive to respiratory and circulatory induced tissue motion.

There is a need for a sensitive, non-invasive method and system for quantifying hemodynamic properties within a living tissue of a subject.

### SUMMARY

In accordance with the present invention, a system and a method are defined for determining hemodynamic properties in a sample of a subject. In one embodiment, the method may comprise performing a plurality of fast scans on a fast scan axis and a plurality of slow scans on a slow scan axis of the sample with a probe beam from a light source, obtaining one or more spectral interference signals from the sample during the plurality of scans, separating the spectral interference signals concerning cell motion within the sample by decomposing the cell motion into orthogonal basis functions, and determining hemodynamic properties of the sample from the spectral interference signals concerning cell motion. In further embodiments, separating the spectral interference signals may further comprise separating spectral interference signals concerning tissue motion and/or noise within the sample by decomposing the tissue motion and/or noise into orthogonal basis functions.

The data from the spectral interference signals concerning cell, tissue, or particle motion within the sample may be extracted using a super-resolution estimation technique, multiple signal classification (MUSIC). The method may be used for diagnosing, providing a prognosis, or monitoring treatment of a disorder of a sample, such as a living tissue in a subject, for example. Particularly, the subject may be at risk of a vascular pathology or has a vascular pathology. The pathology may be but is not limited to one or more of glaucoma, age-related macular degeneration, diabetics, cancer, stroke, and a number of other disorders involving vascular components, for example, disorders of the brain, kidney, and skin. Such assessments may be used to provide guidance in medical, laser, or surgical management for a disorder of the tissue.

In one embodiment, the method may further comprise an ultrahigh sensitive optical microangiography UHS-OMAG imaging protocol to perform the plurality of fast scans on a fast scan axis with the probe beam from the light source, performing a plurality of slow scans on a slow scan axis, and obtaining a data set from the plurality of fast and slow scans.

In another embodiment, a system for determining hemodynamic properties is provided. The system includes an optical coherence tomography probe, an optical circulator, a coupler, a spectrometer, and a physical computer-readable storage medium. The system is configured to acquire images from living tissue. The physical computer-readable storage medium has stored thereon instructions executable by a processor to cause the processor to perform functions to extract microcirculation data from images acquired from optical coherence tomography scans of the tissue, the functions comprising: performing a plurality of fast scans on a fast scan axis and a plurality of slow scans on a slow scan axis of the sample with a probe beam from a light source, obtaining one or more spectral interference signals from the sample during the plurality of scans, separating the spectral interference signals concerning cell motion within the sample by decomposing the tissue motion, the cell motion, and the noise into orthogonal basis functions, and determining hemodynamic properties of the sample from the spectral interference signals concerning cell motion. In further embodiments, separating the spectral interference signals function may further comprise separating spectral interference signals concerning tissue motion and/or noise within the sample by decomposing the tissue motion and/or noise into orthogonal basis functions.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a block diagram of an imaging apparatus in accordance with at least one embodiment;

FIG. 2 depicts an image of a mouse ear pinna flat mounted in accordance with at least one embodiment;

FIG. 3a depicts a MUSIC-OMAG image illustrating lower band power taken with the exemplary system of FIG. 1 for the mouse ear pinna of FIG. 2, in accordance with at least one embodiment;

FIG. 3b depicts a MUSIC-OMAG image illustrating upper band power taken with the exemplary system of FIG. 1 for the mouse ear pinna of FIG. 2, in accordance with at least one embodiment;

FIG. 3c depicts a MUSIC-OMAG image illustrating combined lower band and upper band power from FIGS. 3a and 3b, in accordance with at least one embodiment;

FIG. 3d depicts a UHS-OMAG image corresponding to the MUSIC-OMAG processed image depicted in FIG. 3c, in accordance with at least one embodiment;

FIG. 4a depicts a UHS-OMAG image of a mouse ear pinna taken with the exemplary system of FIG. 1 in accordance with at least one embodiment;

FIG. 4b depicts the MUSIC-OMAG image of the mouse ear pinna from FIG. 4a in accordance with at least one embodiment;

FIGS. 5a-5l depict a series of dynamic images created using a MUSIC-OMAG analysis, in accordance with at least one embodiment;

FIG. 6a depicts a graph illustrating the mean value of the normalized total blood flow plotted as a function of temperature, in accordance with at least one embodiment;

FIG. 6b depicts a graph illustrating normalized vessel area density plotted over temperature values in Celsius, in accordance with at least one embodiment;

FIG. 7a depicts an en-face view of a maximum-intensity map using MUSIC-OMAG quantification of micro-vasculature in the mouse ear pinna of FIGS. 4a-4b, in accordance with at least one embodiment;

FIG. 7b depicts a detail view of an area within the image in FIG. 7a, in accordance with at least one embodiment:

FIG. 7c depicts a graph illustrating three vessel profiles at vessel locations marked from FIG. 7b, in accordance with at least one embodiment;

FIG. 7d depicts a graph illustrating three vessel profiles at vessel locations marked from FIG. 7b, in accordance with at least one embodiment; and

FIG. 8 depicts a comparison image data set that compares MUSIC-OMAG analyzed images with complex autocorrelation (CAC) analyzed images over four data sets from thermoregulatory experiments, in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Embodiments herein combine data acquired using an ultrahigh sensitive optical microangiography (UHS-OMAG) system (that delivers high sensitivity with a relatively low data acquisition time) with a super-resolution estimation technique, such as multiple signal classification (MUSIC), to quantify and visualize hemodynamic properties, such as blood flow in vessels and capillaries and blood flux in the microcirculatory system. Such quantification includes estimating and determining the number of blood cells (e.g., red blood cells) passing through vessels per unit of time. The blood flux measurement allows for estimating the blood perfusion within tissue beds surrounding capillary beds, which is helpful for estimating metabolic activity of a tissue.

The embodiments herein provide for dynamic estimation and separation of moving tissues from stationary tissues, allowing the ability to change the estimation based on updated input signals received.

The embodiments herein can dynamically estimate and separate blood flow from stationary tissue using both amplitude and phase information, rendering the techniques described herein sensitive to both axial and transverse flow.

OMAG is an imaging modality that is a variation on optical coherence tomography (OCT). The imaging is based on the optical signals scattered by moving particles. The light backscattered from a moving particle may carry a beating frequency that may be used to distinguish scattering signals by the moving elements from those by the static elements. Thus, the optical signals backscattered from the moving blood cells are isolated from those originated from the tissue microstructures. Accordingly, OMAG can be used to image the flow of particles, such as blood flow.

FIG. 1 depicts a block diagram of an imaging apparatus 100 in accordance with at least one embodiment. The imaging apparatus 100 may be an SD-OCT apparatus suitable for application with the super-resolution spectral estimation technique, which will be described in further detail below. The illustrated imaging apparatus 100 may include some features known in the art, features which may not be explained in great length herein except where helpful in the understanding of embodiments of present disclosure.

SD-OCT apparatus 100 may be used, among other things, to measure hemodynamic properties of a living tissue sample of a subject. Thus, SD-OCT apparatus 100 may be used on a subject in vivo. As referenced herein, a subject may be a human subject.

As shown in FIG. 1, SD-OCT apparatus 100 includes a light source 110. In one example embodiment, light source 110 comprises a broadband light source, for example, superluminescent diode with a central wavelength of 1310 nanometers (nm) and a full-width-at-half-maximum bandwidth of 65 nm. Light source 110 may give an axial resolution of about 12 μm in the air. In some example embodiments, light source 110 comprises a light source having one or more longer or shorter wavelengths, which may allow for imaging at deeper levels in a sample. In other example embodiments, light source 110 may comprise a tunable laser source, such as, for example, a swept laser source.

Although SD-OCT is used herein to provide an example apparatus that may be used to carry out the methods disclosed herein, the methods disclosed herein are equally applicable to time-domain OCT and swept-source OCT.

SD-OCT apparatus 100 may include optics 111 to couple the light from light source 110 into a fiber-based interferometer 112. In some example embodiments, interferometer 112 may comprise a fiber-based Michelson interferometer, such as a 2×2 fiber coupler.

Interferometer 112 may then split the light into two beams: a first beam provided to a reference arm 113 and a second beam provided to a sample arm 114.

The reference arm 113 may comprise a polarization controller 115 and a reference mirror 116. Reference mirror 116 may be stationary or may be modulated.

Sample arm 114 may comprise a polarization controller 118, a collimating lens 120, an objective lens 122, an X-scanner 124, and a Y-scanner 126. Objective lens 122 may comprise a microscopy objective lens with 18 mm focal length that may be used to achieve about 5.8 μm lateral resolution.

Sample arm 114 may be configured to provide light from light source 110 to a sample 130 by way of lenses 120, 122. X-scanner 124 and Y-scanner 126 may comprise a pair of x-y galvanometer scanners for scanning sample 130 in an x-y direction. In the present example embodiment, a mouse ear pinna was used as a living sample for sample 130, with the goal to visualize and quantify the blood flow within microcirculatory tissue beds.

A laser diode 140 may be used as a guiding beam to locate the imaging position, since the wavelength of the light source 110 is invisible to the human eye. The laser diode 140 may be a 633 nm laser diode, in one example embodiment. Such a guiding beam may help adjust the sample under the OCT system 100 and image the desired location.

The light returning from reference arm 113 and from sample arm 114 may be recombined and coupled into interferometer 112 for introduction to a detection arm 150 via circulator 111. As shown in FIG. 1, detection arm 150 comprises a spectrometer 152 including one or more of various optics including, one or more collimators 154, one or more diffracting/transmission gratings 155, one or more lenses 156, and an InGaAs linescan camera 157. In an exemplary embodiment, collimator 154 comprises a 30 mm focal length collimator. A 14-bit, 1024-pixels InGaAs linescan camera may be used as linescan camera 157, with a camera speed of 47000 lines per second. The spectral resolution of the spectrometer may be about 0.141 nm to provide a detectable depth range of about 3.0 mm on each side of the zero-delay line. The system 100 may have a measured signal to noise ratio of about 105 dB with a light power on the sample 130 at about 3 mW.

The main computing system 158 may be the same as or similar to any number of computing systems known in the art and may include a processor, data storage, and logic. These elements may be coupled by a system or bus or other mechanism. The processor may include one or more general-purpose processors and/or dedicated processors, and may be configured to perform an analysis on the output generated from the line scan cameras in the system 100. An output interface may be configured to transmit output from the computing system 158 to a display.

In some example embodiments, the scanning protocol may be based on a three-dimensional UHS-OMAG technique. X-scanner 124 may perform at least one fast scan along a fast scan axis, and y-scanner 126 may perform at least one slow scan along a slow scan axis. The fast scan axis may be orthogonal to the slow scan axis. The fast scan may also be referred to as the x-axis, the lateral axis, and/or the B-scan axis, and may be driven with a saw tooth waveform. Similarly, the slow scan may also be referred to herein as a C-scan, may also be referred to as the y-axis, the elevational axis, and/or the C-scan axis, and may be driven with a step function waveform. Each fast scan may be performed over a fast scan time interval, and each slow scan may be performed over a slow scan time interval, where the slow scan time interval is at least twice as long as the fast scan time interval. In some embodiments, one or more fast scans may be performed contemporaneously with the one or more slow scans. In such embodiments, a plurality of fast scans may be performed during one slow scan. A combination of slow and fast scans provides a 3D data set necessary to obtain a 3D image. Thus, an imaging protocol comprises a plurality of fast scans on the fast scan axis and a plurality of slow scans on the slow scan axis.

In each B-scan there may be a number of A-scans. In one example embodiment, 400 A-lines covering a range of about 2.22 mm on a sample may be used. Other quantities of A-lines and ranges may be envisioned without deviating from the embodiments as described herein. Similarly, a C-scan may include a number of B-scans.

A B-scan rate of about 94 frames per second may be performed, and a C-scan may comprise 400 scan locations with B-scan repetition of 8 frames per location for flow imaging and quantification, in one example embodiment. Other quantities of frames per second, scan locations, and repetitions per location may be envisioned without deviating from the embodiments as described herein.

In some example embodiments, the super-resolution spectral estimation technique MUSIC may be applied to the data set obtained from a system such as the system 100. MUSIC is a noise subspace frequency estimator based on the principle of orthogonality, wherein noise space eigenvectors of the autocorrelation matrix (i.e., the data matrix) are orthogonal to the signal eigenvectors, or any linear combination of the signal eigenvectors. The frequency resolution of MUSIC is independent of the number of fast Fourier transform (FFT) points, rendering MUSIC a super-resolution method. The MUSIC estimation technique was previously used for signal processing in radar and other industrial applications. MUSIC has not previously been applied to subject analysis, such as for in vivo tissue applications in a subject.

In one example embodiment, a method applying MUSIC comprises modeling OCT measurements at each voxel to be superpositions of tissue signals (stationary and non-moving structure information), hemodynamic signals, and noise (both shot and system noise). These components are independent and can be decomposed into orthogonal basis functions; thus MUSIC has the capability to separate the components.

The interference signal of one A-scan captured in FDOCT can be expressed by:

I(k)=S(k)E_{R}²+2S(k)E_{R}∫_{-∞}^{∞}α(z)cos(2knz)dz+2S(k)E_{R}α(z₁)cos [2kn(z₁−vt)] Equation 1

where I(k) is the light intensity detected at a wavelength with wavenumber of k at time t, E_{R} is the light reflected from the reference mirror, S(k) is the spectral density of the light source used at k, n is the refractive index of the tissue, z is the depth coordinate, α(z) is the amplitude of the backscattered light, z is the depth from which the light back scattered from, and v is the velocity of moving blood cell in a blood vessel which is located at depth z₁.

In Equation 1, the first term is a dc component produced by the light reflected from the reference mirror. The second term is the spatial frequency component of the static tissue sample, which provides static structural information (i.e. morphological features) of the sample. The third term is contributed from moving particles such as red blood cells in the tissue sample. The dc component may be subtracted from the equation by removing a common average from A-lines.

Assuming that the 3D OCT signal at each voxel is given by a complex value x[n], where n corresponds to the temporal sampling at that voxel location, we can decompose x[n] in terms of its exponential basis function, given by:

x[n]=Σᵢ₌₁^{P}αᵢe^{j(nω}^{ₜ}^{+φ}^{ᵢ}⁾ Equation 2

where P is the total number of orthogonal components in the signal, ωᵢ is the angular frequency of each component, and αᵢ and φᵢ are the amplitude and phase of that component, respectively. Then, the autocorrelation function of x[n] is given by:

rₓₓ[k]=E{x[n]x[n−k]}=Σᵢ₌₁^{P}Aᵢe^{jnω}^{ᵢ} Equation 3

where Aᵢ=αᵢ². Based on different autocorrelation lag values for |k|=1, . . . , M, the autocorrelation matrix is given by:

$${R}_{xx} = [ \begin{matrix}{r}_{xx} [ 0 ] & {r}_{xx} [ - 1 ] & … & {r}_{xx} [ - ( M - 1 ) ] \\ {r}_{xx} [ 1 ] & {r}_{xx} [ 0 ] & … & {r}_{xx} [ - ( M - 2 ) ] \\ ⋮ & ⋮ & … & ⋮ \\ {r}_{xx } [ M - 1 ] & {r}_{xx} [ M - 2 ] & … & {r}_{xx} [ 0 ]\end{matrix} ] \qquad Equation 4$$

where M is the number of temporal samples.
If M>p (acquiring more samples than the number of signal components), then:

Rank{Rₓₓ}=min{M,P}=P. Equation 5

the eigenvalues of Rₓₓ may be characterized as λ₁≧λ₂≧λ₃≧ . . . ≧λ_{M} corresponding to the normalized eigenvectors u₁, u₂, . . . , u_{M}. Then, the eigendecomposition of Rₓₓ may be:

Rₓₓ=Σᵢ₌₁^{M}λᵢuᵢuᵢ^{H}. Equation 6

Since Rₓₓ is of rank P, then λₚ₊₁=λₚ₊₂= . . . =λ_{M}=0, and Rₓₓ can be represented by its first P eigenvalues and eigenvectors given by:

Rₓₓ=Σᵢ₌₁^{P}λᵢuᵢuᵢ^{H}. Equation 7

wherein the eigenvectors {u₁, u₂, . . . , u_{P}} are the principal eigenvectors of autocorrelation matrix Rₓₓ that spans the signal subspace.
The autocorrelation matrix may be represented as:

Rₓₓ=Σₖ₌₁^{p}Aₖsₖsₖ^{H}=SAS^{H} Equation 8

where S_{M×P}=[S₁ S₂ . . . Sₚ], Sᵢ=[1 e^{jω}^{ᵢ} e^{j2ω}^{ᵢ} . . . e^{j(M-1)ω}^{ᵢ}]′, and A=diag([A₁, A₂, . . . , Aₚ]). H is the matrix Hermitian (complex conjugate transpose) and diag([.]) is a diagonal matrix. The vector space S_{M×P}={S₁, S₂, . . . Sₚ} may be called the signal subspace of {x[n]}.

Based on noise subspace principles, a frequency estimator function can be developed that exhibits pseudo-spectrum plots with sharp peaks. Theoretically, the M−P noise subspace eigenvectors (u_{P+1}, u_{P+2}, . . . , u_{M}) of the autocorrelation matrix of M total eigenvectors and P principle eigenvectors ({u₁, u₂, . . . , u_{P}}) will be orthogonal to the sinusoidal signal subspace vector (S). Therefore, a linear combination with an arbitrary weighting αₖ may be given by:

Σ_{k=P+1}^{M}αₖ|S^{H}(ω)uₖ|²=S^{H}(ω)(Σ_{k=P+1}^{M}αₖuₖuₖ^{H})S(ω) Equation 9

where S(ω)=[1 e^{jω} e^{j2ω} . . . e^{j(M-1)ω}]′ is the sinusoidal vector that would be zero if evaluated at S(ωᵢ)=Sᵢ, one of the input sinusoidal signal frequencies. Therefore, the MUSIC spectral estimator function:

$$P ( ω ) = \frac{1}{{\sum }_{k = p + 1}^{M} {\vert {S}^{H} ( ω ) {u}_{k} \vert}^{2}} \qquad Equation 10$$

will theoretically have an infinite value if evaluated at one of the sinusoidal signal frequencies (ω=ωᵢ). In practice, the MUSIC frequency estimation function is finite due to estimation error, but exhibits a local maximum (i.e. a peak) at the sinusoidal frequencies. Locating the peak and its corresponding value will be an indicator of the hemodynamics at the voxel of interest.

The backscattered OCT signal has relatively higher signal-to-noise ratio (SNR) at stationary and non-moving tissue boundaries because the structure pattern is repeatable. However, the backscattered signal from moving scatterers such as moving red blood cells inside patent vessels is typically weaker and temporally varying. Since the tissue component is stronger than the hemodynamic component, their corresponding MUSIC eigenvalues will be in order so that the larger eigenvalue belongs to tissue signal subspace while smaller eigenvalue belongs to the hemodynamic signal subspace. Therefore, their corresponding subspaces can be separately estimated.

In MUSIC, the number of input signal components (P) is a user-defined input variable. By defining the number of input signal components to be P=2, the largest peak in the MUSIC pseudo-spectrum of UHS-OMAG data corresponds to the stationary tissue and the second largest peak corresponds to the hemodynamics. We can also approach this problem by first removing the stationary and non-moving tissue structural components (also known as clutter) from the input data, and then characterizing the remaining component which corresponds to the hemodynamics. This can be done using eigendecomposition-based clutter rejection filtering technique, which is performed on repeated A-lines at the same spatial location.

Multiple A-lines are acquired from the same location. After removing the dc component in Equation 1, the phase difference at each depth location is utilized to estimate its corresponding average flow velocity. The received backscattered signal at a particular depth along each A-line form a vector defined as follows:

X=[x(1),x(2), . . . ,x(N)]^{T} Equation 11

where N is the ensemble size. The observation or ensemble of samples from one particular depth location is modeled as the sum of three independent zero-mean complex Gaussian processes: a clutter component c, a blood component b, and additive white noise n. Its vector notation is given by:

X=c+b+n Equation 12

ED-based filtering takes advantage of the characteristics unique to high-frequency blood flow mapping, such as that tissue motion is correlated over the depth of interest and tissue motion velocities are small but on the same order of the blood flow velocity.

Since X is Gaussian, it is characterized by its correlation matrix Rx, given by:

Rx=Rc+Rb+σ²ₙI Equation 13

where R_{c} is the clutter correlation matrix, R_{b} is the blood correlation matrix, σ²ₙ is the noise variance, and I is the identity matrix.

Assuming that clutter is the dominant signal and its characteristics are similar along the depth, the spatial average of the correlation of the received signal along the axial direction is an estimate of the clutter correlation matrix R_{c} given by:

$${R}_{c} = \frac{1}{M} {\sum }_{i = 1}^{M} {R}_{c} = \frac{1}{M} {\sum }_{i = 1}^{M} {X}_{i} {X}_{i}^{H} \qquad Equation 14$$

where Xᵢ is the complex Doppler signal from depth i, and (.)^{H} is the Hermitian transpose. The estimated correlation matrix Rc is decomposed into its corresponding eigenvalues and eigenvectors given by:

Rc=EΛE^{H} Equation 15

where E=[e₁, e₂, . . . , e_{N}] is the N×N unitary matrix of eigenvectors, Λ=diag {λ₁, λ₂, . . . , λ_{N}} is the N×N diagonal matrix of eigenvalues and λ₁≧λ₂≧ . . . λ_{N}=σ²ₙ, and σ²ₙ is the noise variance. Assuming that the clutter space is spanned by K eigenvectors, an eigenregression filter is applied to the received signal by removing the clutter components as follows:

Y=(l−Σᵢeᵢeᵢ^{H})X Equation 16

where Y is the Doppler signal after removing the clutter component. Also, the corresponding frequency response of this filter can be represented by:

$$H ( ω ) = 1 - \frac{1}{N} {\sum }_{i} {\vert D T F T \{ {e}_{i} \} \vert}^{2} \qquad Equation 17$$

where DTFT is the discrete-time FT (DTFT).

The Doppler center frequency of the flow is estimated by:

$$fb = 1 \frac{1}{2 π} atan ( \frac{Im \{ Ry ( 1 ) \}}{( Re \{ Ry ( 1 ) \}} ) \qquad Equation 18$$

where Ry (1) is the first lag autocorrelation of Y.

The advantage of this approach is that after removing the clutter, a mask image based on the remaining flow information can be created and MUSIC is performed only on the voxels with high flow value, which would dramatically reduce the total processing time.

To further measure the blood flux and flow, a scanning protocol based on wide velocity range Doppler microangiography may be used prior to analysis using the equations described above. In this example scanning protocol, the probe beam is shifted to each spatial location and after the scanner is stabilized, multiple repeated A-scans per location are captured at a defined scan frequency (defined by Nyquist rate). Then, the probe beam is shifted to the adjacent spatial location and the same procedure continues until all the locations in the field of view on the tissue are covered. The advantage of this method is that temporal power spectral density broadening due to the moving scanner speed is minimized because the scanner is fully stabilized. In one example embodiment, 25 A-lines may be acquired in repetition per location, 200 A-lines per B-frame, and 200 frames for each 3D scan. Other quantities of A-lines and frames may be envisioned without deviating from the embodiments as described herein. Since in this example embodiment, the camera is triggered at the defined scan frequency of 7 kHz (due to the Nyquist rate), the total scanning time for a 3D data set is about 140 seconds.

### Example 1

### Imaging and Assessment of a Mouse Ear Pinna In Vivo

In one example procedure, non-invasive in vivo images were acquired from pinna of healthy about eight week old male hairless mice weighing approximately 28 grams (g) and were analyzed using the MUSIC technique. The procedure and results are described in S. Yousefi et al., Super-Resolution Spectral Estimation of Optical Micro-Angiography for Quantifying Blood Flow within Microcirculatory Tissue Beds In Vivo, Biomedical Optics Express, Jun. 27, 2013, which is incorporated herein by reference in its entirety.

During the experiments, each mouse was anesthetized using 2% isoflurane, and the mouse ear was kept flat on a microscope glass. The mouse was placed in supine position on a heating blanket using an intra-rectal temperature by the use of temperature feedback provided by the heating blanket.

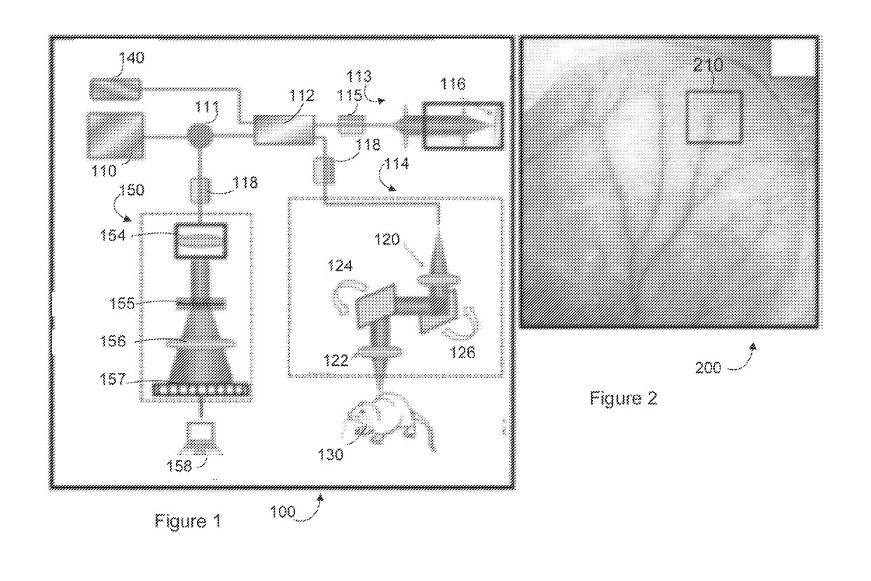

FIG. 2 depicts an image 200 captured by a digital camera of the mouse ear pinna flat mounted, as described above. The rectangle 210 shows a typical OCT imaging field of view and scanning range, representing 2.2×2.2 mm². To scan a larger field than the field represented by the rectangle 210, a mechanical translating stage may be used to move the tissue sample and after acquisition and processing of the images, the images can be stitched together to form a larger image.

Using the scanning protocol discussed above, with a B-scan frame rate of about 94 frames per second and a C-scan of 400 scan locations with B-scan repetition of 8 frames per location, the total size of the data set comprised 1.28×10⁶ A-lines and a total acquisition time of 32 seconds. The captured data was then processed using MUSIC-OMAG visualization.

For MUSIC-OMAG visualization, a dynamic range of MUSIC power (P(ω)) was provided as two bands: lower band power and upper band power, which are separated using a threshold value. In some embodiments, the threshold may be manually set to a value such that the blood flow in small vessels and capillary loops are separated from that of the larger vessels. The threshold value may be variable depending on the sampling rate and the structure to be imaged. A user may arbitrarily choose the threshold value based on the characteristics that are desired to be emphasized. The threshold value is used for visualization purposes and does not impact the quantification of the blood flow.

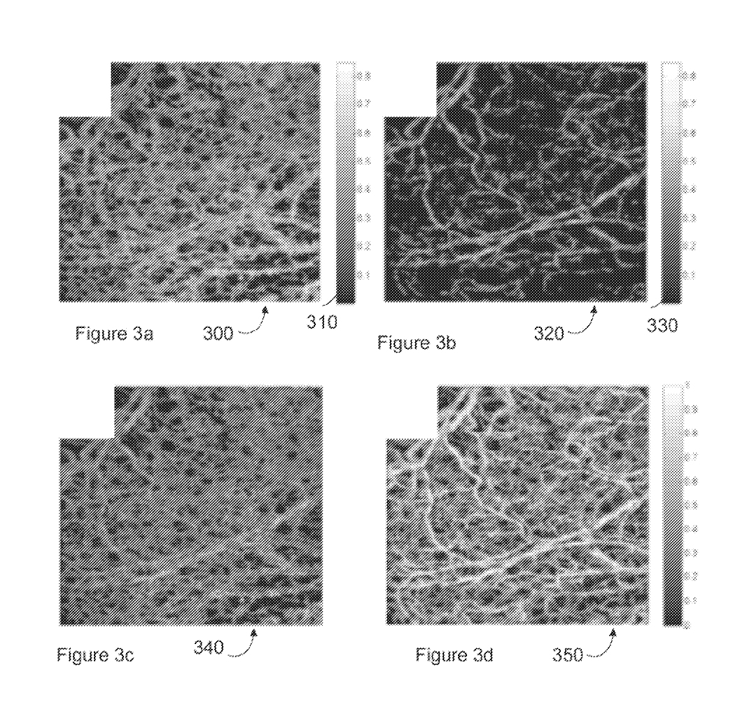

FIG. 3a is a MUSIC-OMAG image 300 depicting lower band power 310. FIG. 3b is a MUSIC-OMAG image 320 depicting upper band power 330. FIG. 3c is a MUSIC-OMAG image 340 depicting combined lower band and upper band power. In FIGS. 3a-3c, the threshold value was set such that the lower band power 310 corresponds to the slower flow inside small vessels and capillary loops.

FIG. 3d is a corresponding UHS-OMAG image 350 corresponding to the MUSIC-OMAG processed image 340 depicted in FIG. 3c. A comparison of the UHS-OMAG image 350 and the MUSIC-OMAG image 340 shows they are almost identical and that the small vessels and capillaries observed in the UHS-OMAG image 350 can also be found in the MUSIC-OMAG image 340, confirming the sensitivity of MUSIC-OMAG quantification.

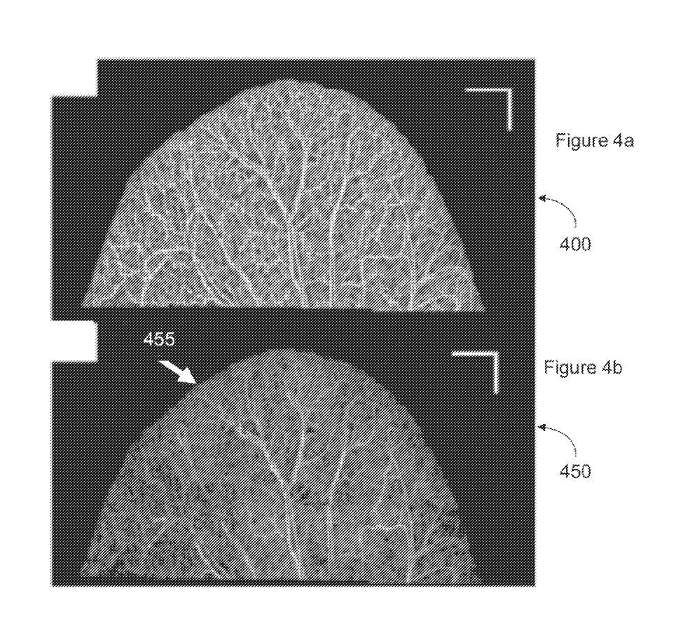

FIG. 4a depicts a UHS-OMAG image 400 of a mouse ear pinna. The entire mouse ear pinna was divided into 2.2×2.2 mm² overlapping mosaics and each mosaic was scanned using UHS-OMAG scanning protocol. The mosaics were also processed separately using ED-OMAG and MUSIC, and their corresponding maximum intensity projection maps were stitched together to form the entire ear pinna en-face angiogram.

FIG. 4b depicts a MUSIC-OMAG image 450 of the mouse ear pinna from FIG. 4a. In image 450, the larger arteries and veins are dominated by upper band power while smaller vessels and capillary loops toward the pinna edge 455 are mainly dominated by lower band power. The quantification and visualization technique of MUSIC-OMAG allows for observation of a certain response in capillary loops while the change in larger vasculature is not significant.

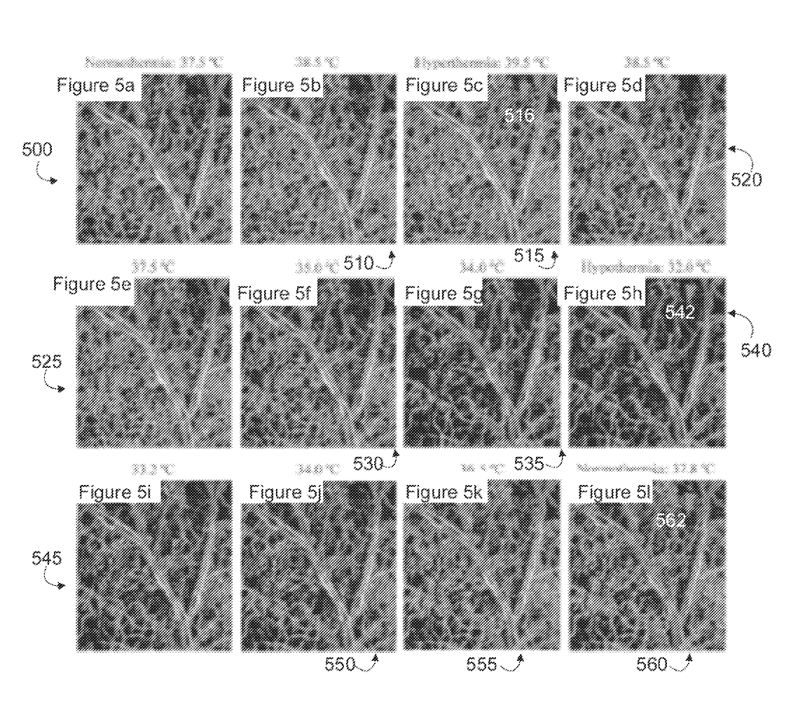

FIGS. 5a-5l depict a series of dynamic images 500-555 created using a MUSIC-OMAG analysis. The images 500-555 were captured by taking active feedback of the body temperature of the sample used. In images 500-555 the response of the capillary flow to various temperature changes is observed.

Over a time period of sixty (60) minutes, the sample's body temperature was actively maintained by a heating blanket while an OCT system, such as the system 100 of FIG. 1, continuously captured a UHS-OMAG dataset. The UHS-OMAG dataset may be captured as described above. MUSIC-OMAG analysis was then used on the UHS-OMAG dataset to determine hemodynamic functions.

As shown in FIGS. 5a-5l, the temperature of the sample's body was gradually raised from 37° C. (FIG. 5a) to 39.5° C. (FIG. 5c) and then followed by a gradual drop to 32° C. (FIG. 5h) before returning to 37.8° C. (FIG. 5l), the target temperature for normal physiological condition. Microcirculation responses to such changes in body temperature were monitored, proving the sensitivity of MUSIC-OMAG to capillary hemodynamic variations.

The increase of body temperature towards hyperthermia (39.5° C.) leads to an increase of the density of capillary network in the areas between larger vessels. Additionally, new capillaries appear as shown by arrows 516 in FIG. 5c. This demonstrates the increase of blood flow within microcirculatory tissue beds during hyperthermia.

The decrease of body temperature towards hypothermia (32.0° C.) showed that most of the small capillaries disappeared and blood flow in some larger vessels also decreased, as indicated by arrows 542 in FIG. 5h.

During the increase of body temperature towards normothermia (37.8° C.), the functional capillaries which were missing in hyperthermia appeared again, as shown in FIG. 5l. At this point, the appearance of the blood vessel network was very similar to the baseline image at 37.5° C., as indicated by arrows 562, for example.

Mean and standard deviation of total blood flow were measured for FIGS. 5a-5l. Then, the mean value was normalized by the total blood flow in the beginning of normothermia.

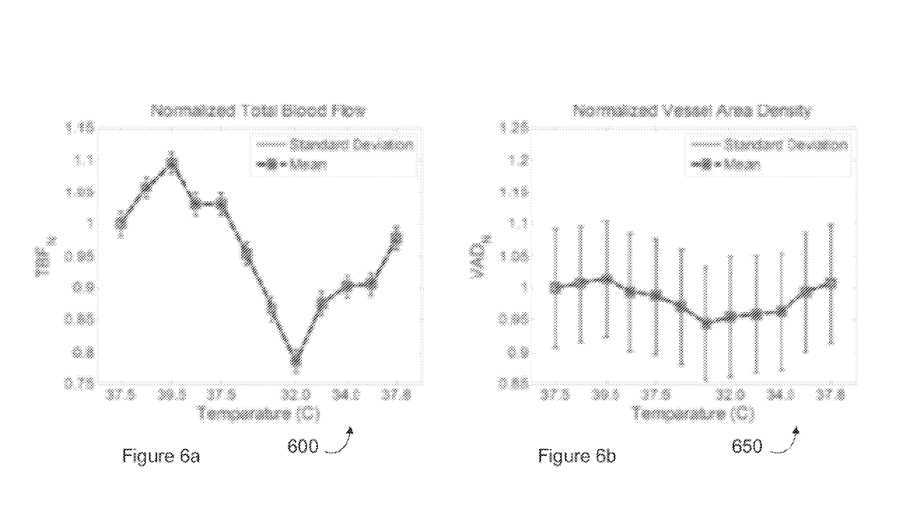

FIG. 6a depicts a graph 600 illustrating the mean value of the normalized total blood flow plotted as a function of temperature values in Celsius. As shown in graph 600, the total blood flow increased during hyperthermia, decreased during hypothermia, and almost went back to the baseline. FIG. 6b depicts a graph 650 illustrating normalized vessel area density plotted over temperature values in Celsius.

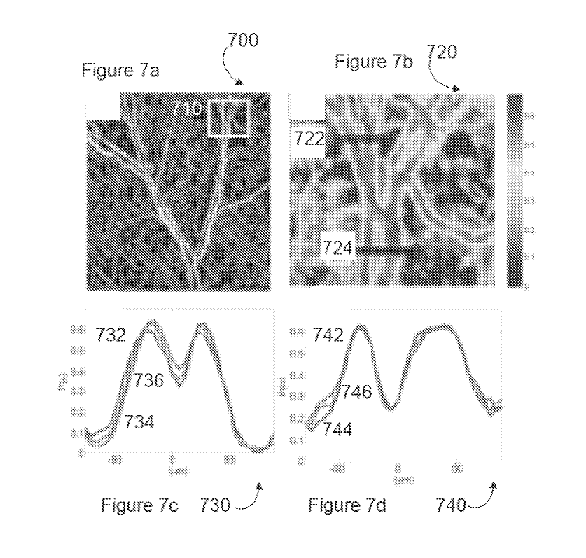

FIG. 7a depicts an en-face view of a maximum-intensity map 700 of MUSIC-OMAG quantification of micro-vasculature in the mouse ear pinna of FIGS. 4a-4b in a 2.2×2.2 mm² area using the threshold visualization technique discussed above with reference to FIG. 4a. A rectangle 710 is depicted in map 700.

FIG. 7b depicts a detail view 720 of an area within the rectangle 710 of FIG. 7a.

Normal blood flow inside relatively large blood vessels is generally not uniform at the vessel cross-section and has a parabolic distribution with the maximum value at the center of the vessel, then trending slower towards the vessel wall. Parabolic or laminar flow allows minimum loss kinetic energy and fluid pressure transfer and reduces friction by allowing the blood layers to slide smoothly over each other in concentric layers or laminae. Therefore, a parabolic quality is expected across the vessel cross-section after quantifying the flow using MUSIC-OMAG.

FIG. 7c depicts a graph 730 illustrating three vessel profiles at vessel location marked by line 722 from FIG. 7b. In the graph 730, the estimated MUSIC-OMAG power (P(ω)) in normalized units is plotted over the horizontal line in μm. Three consecutive locations were plotted to confirm their similarities along the vessels and repeatability of MUSIC-OMAG; thus line 732 corresponds to a first plot, line 734 to a second plot, and line 736 to a third plot.

FIG. 7d depicts a graph 740 illustrating three vessel profiles at vessel location marked by line 724 from FIG. 7b. In the graph 740, the estimated MUSIC-OMAG power (P(ω)) in normalized units is plotted over the horizontal line in μm. Three consecutive locations were plotted to confirm their similarities along the vessels and repeatability of MUSIC-OMAG; thus line 742 corresponds to a first plot, line 744 to a second plot, and line 746 to a third plot.

From graphs 730 and 740, it is observed that the flow profile meets a typical laminar flow profile, such as that described above, inside vessels where the flow value is largest in the middle of the vessel and decreases towards the vessel wall. The blood flow is nearly zero outside vessels where no flow exists.

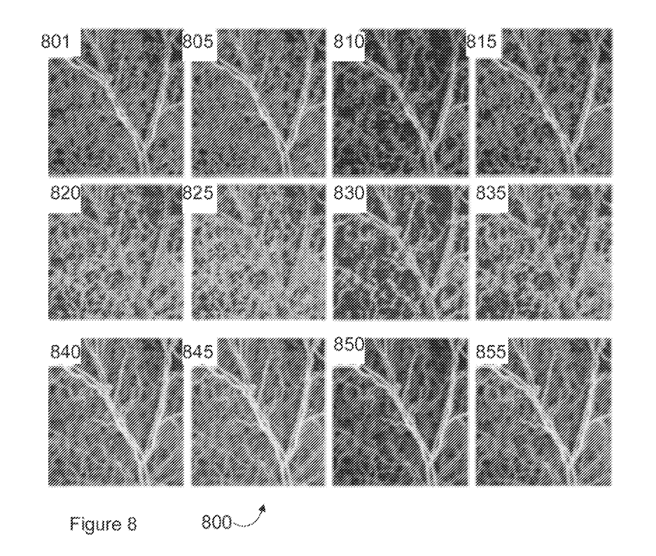

FIG. 8 is a comparison image data set 800 depicting images 801-855 that compares MUSIC-OMAG analysis with a complex autocorrelation (CAC) method over four data sets from thermoregulatory experiments: normothermia (37.8° C.) at images 801, 820, and 840, hyperthermia (39.5° C.) at images 805, 825, and 845, hypothermia (32.0° C.) at images 810, 830, and 850, and return to normothermia (37.5° C.) at images 815, 835, and 855.

Images 801-815 depict the MUSIC-OMAG analysis for each temperature datapoint. Images 820-835 depict the CAC analysis for each temperature datapoint. Images 840-855 depict a corresponding UHS-OMAG processing for each temperature datapoint.

CAC utilizes OCT complex signals instead of only the amplitude information of an OCT signal. The widening of the bandwidth in the power spectral density of autocorrelation function of the input data around Doppler frequency is estimated, allowing for the estimation of absolute blood flow velocity in capillaries and vessels. CAC requires the capturing of a large data set, which translates to a long acquisition time, is sensitive to tissue motion, exhibits artifacts at the vessel boundaries, and aliasing in vessels with fast flow rates.

As shown in images 820-835, the CAC analysis was capable of picking up changes in capillary blood flow; however, CAC analysis was sensitive to tissue motion. Thus, compared to UHS-OMAG and MUSIC-OMAG, the CAC analysis eventually produced vertical stripes due to tissue motion on its images. The CAC analysis also produced some artifacts on vessel walls which are not observed in the MUSIC-OMAG images 801-815. The signal in the CAC analysis images 820-835 inside the large vessels was aliased due mainly to fast flow relative to the Nyquist rate, and the received signal at that location was also decorrelated; these issues are not observed in the MUSIC-OMAG images 801-815. The performance of CAC analysis depends on the sampling rate (to avoid aliasing) and the number of data points (frequency resolution). Since the signal at each voxel decorrelates between images, the dynamic range of a CAC analysis is relatively small.

The MUSIC-OMAG images 801-815 are observed to be sensitive to small capillary response due to a change of body temperature. The flow profile at large vessels evaluated by MUSIC-OMAG is in agreement with typical flow characteristics.

Thus, the comparison images depicted in FIG. 8 shows that the performance of MUSIC-OMAG is superior to CAC.

An OCT system such as the system 100 to capture a dataset, followed by MUSIC analysis of the dataset, allows for the quantification of hemodynamics in micro-vessels. Such an ability opens a new realm of possibilities for diagnosing, monitoring, and therapeutic guidance in the management of disease processes of glaucoma, cancer, stroke, and a number of other disorders involving vascular components, for example, disorders of the brain, renal region, and skin. Such assessments may be used to provide guidance in medical, laser, or surgical management for a disorder.

The system 100 may be a useful tool for the study of mechanisms associated with regulation of blood flow, effects of pharmacologic agents and vascular components of pathologic processes associated with a number of tissue disease states. The system 100 may be used for a subject at risk of any pathology involving vascular components, including but not limited to glaucoma, cancer, stroke, diabetes, age-related macular degeneration, diabetic retinopathy, vasculitis, angioneurosis, neurovascular and retinal disease, disorders of the brain, disorders of the renal region, and disorders of the skin.

The determination of microvascular functions may be used to diagnose, provide a prognosis, monitor treatment and guide treatment decisions for a disorder of the sample of a subject. The treatment may include medical, laser, or surgical intervention. A treatment decision may be based on the prognosis, monitoring or assessment of current properties of the tissues or regions of the tissue conducted in accordance with the determination of microvascular functions performed in the manner described above.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

1. A method for determining hemodynamic properties in a sample of a subject comprising:
performing a plurality of fast scans on a fast scan axis and a plurality of slow scans on a slow scan axis of the sample with a probe beam from a light source;
obtaining one or more spectral interference signals from the sample during the plurality of scans;
separating the spectral interference signals concerning cell motion within the sample by decomposing the cell motion into an orthogonal basis function; and
determining hemodynamic properties of the sample from the separated cell motion spectral interference signals.

2. The method of claim 1, wherein performing the plurality of fast scans on the fast scan axis and the plurality of slow scans on the slow scan axis comprises an ultrahigh sensitive optical microangiography (UHS-OMAG) imaging protocol.

3. The method of claim 1, wherein separating the spectral interference signals further comprises applying both amplitude and phase data to separate interference signals concerning tissue motion from stationary tissue.

4. The method of claim 1, further comprising:
applying a threshold value to separate cell motion in small vessels from cell motion in large vessels; and
producing a first image depicting blood flow from the cell motion in the small vessels and a second image depicting blood flow from the cell motion in the large vessels.

5. The method of claim 1, wherein determining hemodynamic properties of the sample from the separated cell motion further comprises:
monitoring microcirculation responses to physiological variations in the subject.

6. The method of claim 1, wherein determining hemodynamic properties of the sample from the separated cell motion further comprises:
generating a flow profile from the separated cell motion per unit area within the sample.

7. The method of claim 1, wherein the hemodynamic properties of the sample includes one or more measurements of a number, a concentration, and a velocity of cell particles per unit area of the sample.

8. The method of claim 7, further comprising:
determining a cell flux and flow from the measurements of the number, the concentration, and the velocity of cell particles per unit area in the sample.

9. The method of claim 1, further comprising:
assessing one or more of tissue perfusion, an oxygen exchange rate, and a nutrition exchange rate within a microstructure.

10. The method of claim 9, further comprising:
estimating metabolic activity of a tissue from one or more of the assessments.

11. The method of claim 1, wherein the subject is at risk of or has one or more disorders selected from the group consisting of glaucoma, age-related macular degeneration, diabetes cancer, stroke, brain disorders, renal disorders, and skin disorders.

12. The method of claim 1, wherein the method is used to diagnose, provide a prognosis, monitor treatment, or provide guidance in medical, laser or surgical management for a disorder involving vascular components of a living tissue.

13. The method of claim 1, wherein the method is used to measure blood perfusion.

14. A system for measuring hemodynamic properties comprising:
an optical coherence tomography probe;
a coupler to receive light emitted from the optical coherence tomography probe;
a spectrometer to receive light split by the coupler; and
a physical computer-readable storage medium;
wherein the system is configured to acquire images from living tissue;
wherein the physical computer-readable storage medium has stored thereon instructions executable by a processor to cause the processor to perform functions to extract microcirculation data from images acquired from optical coherence tomography scans of the tissue, the functions comprising:
performing a plurality of fast scans on a fast scan axis and a plurality of slow scans on a slow scan axis of the sample with a probe beam from a light source;
obtaining one or more spectral interference signals from the sample during the plurality of scans;
separating the spectral interference signals concerning cell motion within the sample by decomposing the cell motion into an orthogonal basis function; and
determining hemodynamic properties of the sample from the separated cell motion spectral interference signals.

15. The system of claim 14, wherein the function of separating the spectral interference signals further comprises applying both amplitude and phase data to separate tissue motion from stationary tissue.

16. The system of claim 14, the functions further comprising:
applying a threshold value to separate cell motion in small vessels from cell motion in large vessels; and
producing a first image depicting blood flow from the cell motion in the small vessels and a second image depicting blood flow from the cell motion in the large vessels.

17. The system of claim 14, the function of determining hemodynamic properties of the sample from the separated cell motion further comprising:
generating a flow profile from the separated cell motion per unit area within the sample.

18. The system of claim 14, wherein the hemodynamic properties of the sample includes one or more measurements of a number, a concentration, and a velocity of cell particles per unit area of the sample.

19. The system of claim 18, the functions further comprising:
determining a cell flux and flow from the measurements of the number, the concentration, and the velocity of cell particles per unit area in the sample, and/or estimating metabolic activity of a tissue from one or more of the assessments.

20. (canceled)

21. The system of claim 14, further comprising a laser diode that emits a guiding beam to locate an imaging position.
